(19) United States
(12) Patent Application Publication
(10) Pub. No.: US 2004/0067197 A1
(43) Pub. Date: Apr. 8, 2004

(54) Radiolabeled DNA carrier, method of preparation and therapeutic uses thereof

(76) Inventors: Guy Leclerc, Quebec (CA); Remi Martel, Quebec (CA)

Correspondence Address: David S. Resnick, Nixon Peabody LLP, 101 Federal Street, Boston, MA, 02110-1832, US

(21) Appl. No.: 09/775,479

(22) Filed: Feb. 2, 2001

Related U.S. Application Data
(63) Continuation-in-part of application No. 09/318,106, filed on May 24, 1999, now abandoned.
Application No. 09/318,106 is a continuation of application No. PCT/CA97/00892, filed on Nov. 26, 1997.

(51) Int. Cl. A61K 51/06; A61K 48/00; C07H 21/04

(52) U.S. Cl. 424/1.73; 514/44; 534/10; 536/23.1

(57) ABSTRACT

The present invention relates to a radiolabeled DNA carrier, a method of preparation thereof and the therapeutic uses of this substance to prevent uncontrolled cellular proliferation. The invention also relates to devices incorporating the above radiolabeled DNA carrier (such as an oligonucleotide) for the therapeutic treatment of uncontrolled cellular proliferation. More specifically, the present invention is concerned with the prevention of restenosis by intravascular delivery of radiolabeled DNA carrier at a dilatation site of an artery. This invention is also directed to a method of treatment of vascular proliferative diseases and/or other proliferative disorders such as cancer and related metastasis. More particularly, the invention relates to the preparation of DNA sequences carrying one or several radioisotopes, located within the DNA sequence, and which are able to prevent cell proliferation in vitro and, pursuant to local drug delivery and/or systemic drug delivery, are able to prevent cell proliferation in vivo, more particularly restenosis and malignant tumors. In other words, the invention relates to the synthesis process, the stability data of the radiolabeled DNA carrier, the efficacy of the invention in vitro, in cell culture, and the in vivo delivery of the molecule.

### RELATED APPLICATION

This application is a continuation in part application of U.S. Ser. No. 09/318,106 filed May 24, 1999, now pending, the content of which is entirely incorporated herein by reference.

### BACKGROUND OF THE INVENTION

(a) Field of the Invention

The invention relates to a radiolabeled DNA carrier, a method of preparation thereof and the therapeutic uses of this substance to prevent uncontrolled cellular proliferation. The invention also relates to devices incorporating the above radiolabeled DNA carrier for the therapeutic treatment of uncontrolled cellular proliferation. More specifically, the present invention is concerned with the prevention of restenosis by intravascular delivery of radiolabeled DNA oligonucleotide at a dilatation site of an artery. This invention is also directed to a method of treatment of vascular proliferative diseases and/or other proliferative disorders such as cancer and related metastasis. More particularly, the invention relates to the preparation of DNA sequences carrying one or several radioisotopes, located within the DNA sequence, and which are able to prevent cell proliferation in vitro through local delivery of radioactivity and, pursuant to local drug delivery and/or systemic delivery, are able to prevent cell proliferation in vivo, more particularly restenosis and malignant tumors. In other words, the invention relates to the synthesis process, the stability data of the radiolabeled DNA oligonucleotide, the efficacy of the invention in vitro, in cell culture, and the in vivo delivery of the molecule.

(b) Description of Prior Art

Despite the favorable impact of balloon angioplasty on the non-surgical treatment of coronary and peripheral vascular disease, this otherwise invaluable intervention remains plagued by a high incidence of restenosis that has resisted all pharmacological attempts to prevent it. Proliferation of vascular smooth muscle cells is considered to represent the fundamental process underlying restenosis. Endovascular irradiation, either catheter or stent based, was recently proposed to be used following balloon angioplasty as it is recognized that ionizing radiation may be effective in inhibiting exuberant wound healing responses in various clinical situations. Radiation emitted from a ¹⁹²iridium source (beta and gamma emission), a ⁹⁰yttrium source, or a ⁹⁰strontium/yttrium source (both pure beta emitters) introduced for a short period of time via a catheter system and immediately removed after angioplasty inhibited subsequent intimal hyperplasia in a pig model of restenosis. Similar results were recently obtained, in applicant's laboratory, using radioactive, ³²P ion implanted stents in coronary arteries (Rivard A et al., 1996 , Circulation, 94(8):210). It was suggested that radiation originating from the beta-emitting wires may have inhibited hyperplasia by either killing progenitor cells or by limiting their replicative capacity. Therapies based on 32 phosphorus systemic injections are currently used to treat diseases such as polycythemia vera, chronic myelocytic and lymphocytic leukemias and skeletal metastases of various origins.

Percutaneous transluminal angioplasty is an accepted form of treatment of coronary and peripheral vascular disease. Since its introduction in 1977 for the treatment for coronary disease, primary success rates have reached very high levels (90% to 95%) and complication rates of 1% to 5% are now the standards. But it was observed that in a certain percentage of patients treated by balloon angioplasty the narrowing treated would reoccur at the same site within three to six months. Angiographic studies indicate that the incidence of restenosis following successful balloon angioplasty may be as high as 55% and 65% in the coronary and peripheral arteries respectively. All pharmacological approaches to prevent the occurrence of restenosis have failed. A number of mechanical alternatives to balloon angioplasty have been developed and investigated, and none has yet been shown to diminish conclusively the incidence of restenosis following percutaneous revascularization, except for a modest reduction obtained with the Palmaz-Schatz stent in selected patients. This effect is explained by the propensity of the stent to achieve a consistently greater increase in lumen diameter immediately after the procedure by limiting the phenomenon of elastic recoil. Although many of the risk factors for restenosis have been identified, most of them are difficult to influence.

Percutaneous transluminal angioplasty results in unavoidable vessel wall injury. Disruption of endothelial and vessel wall structure triggers molecular and cellular events that lead in some patients to restenosis. Several growth factors, cytokines and cell-surface receptors have been implicated in this proliferation process. In animal models of vascular injury, following the immediate loss of lumen diameter accounted by elastic recoil, an important cascade of events leads to smooth muscle cell (SMC) proliferation that begins 24 hours post-angioplasty. SMC proliferation appears to be a consistent response to balloon dilatation and/or denudation of the artery. Cell replication has been reported to peak within seven days after the angioplasty; and twenty-eight days after the angioplasty, SMC proliferation in the media as well as in the intima appears normalized. This process is then followed by matrix deposition over the next several weeks.

The same problems are faced in the treatment of localized cancerous tumors where it has been found impossible to achieve a successful localized radiation treatment.

Recent studies have indicated that restenosis rates in animal models can be reduced by intra-coronary radiotherapy (ICRT) during or after angioplasty with or without the application of a stent. Early experiments with low level radioactive stainless steel stents implanted in rabbits indicated a complete inhibition of neointimal cell proliferation (U.S. Pat. Nos. 5,059,166 and 5,176,617). Inhibition of neointimal proliferation has also been recently reported in swine experiments using 14 titanium stents, 7 of which were heavily implanted with ³²P and neutron activated to produce low dose true beta particle emitting isotope phosphorus 32 (³²P).

Intracoronary irradiation with ¹⁹²Ir ribbons has also been reported to markedly reduce neointima formation in swines. ¹⁹²Ir decays via negative beta emission with a maximum energy of 0.67 MeV and primary gamma emission in the energy range of 0.3-0.6 MeV. The half-life is 74.2 days. It is used primarily in brachytherapy where the radioactive source is placed directly into a tumor or at the extirpation site to deliver a given treatment dose and then is removed. This benefit appeared to be sustained at 6 months, with no evidence of late radiation sequelae.

Waksman reported their experience with a high activity ¹⁹²Ir source introduced into a pig coronary arteries after the injury was created (Waksman R. et al., 1995, Circulation, 91:1533-1539). They showed that the intimal area-to-medial fracture length was inversely correlated with the different radiation doses, with a significant reduction in neointimal formation at all doses when compared to control arteries. A report of stent implantation combined with ¹⁹²Ir ribbon radiation dose delivery also indicated a net reduction of neointima formation in pig coronary arteries.

Finally, the use of a wire or catheter with a pure beta emitter (⁹⁰yttrium source), (⁹⁰Strontium/Yttrium source), was reported (Verin et al., 1995, Circulation, 92:2284-2290; Waksman R. et al., 1995, Circulation, 92:1383-1386). A dose-response relation was demonstrated, without further inhibitory effect at doses beyond 28 Gy.

Recently, precision dose of radiation therapy was delivered percutaneously into the human coronary artery in 10 patients. An Iridium-192 source wire was after loaded through a coronary catheter system and a treatment dose of 2 000 cGy was delivered to the intima of each coronary artery segment with treatment times ranging from 5 to 15 minutes. No angiographic control data at 6 month was reported. From this study, it was concluded that percutaneous transluminal coronary angioplasty (PTCA)-ICRT can be delivered safely to humans, but the efficacy of ICRT in reducing coronary restenosis in the test population remains to be asserted.

All of these results are very encouraging and suggest that a significant reduction in the rate of restenosis may be obtained by ICRT following PTCA. However, there is currently no consensus on the ideal radioactive source, the dose delivery level and method to be used.

It would be highly desirable to be able to effect radiotherapy from within the target tissue itself instead of extratissular exposure. It would permit to reduce the dose-gradient to achieve radiation delivery to the medial-adventitial border and thus permit a reduction in total activity necessary to deliver the appropriate dose. Also it may minimize the damage incurred by endothelial cells and thus reduce the risk of thrombotic event by improving healing of the vessel wall. Finally, by minimizing endoluminal exposure to radiation, it may reduce the edge effect related to radiation. This may result in a more efficient strategy to prevent restenosis and potentially any other vascular proliferative disease or/and treat other proliferative processes such as cancer and related metastasis.

In order to effect intra-tissular delivery of radioactivity, the vector of radiation should be a molecule that can be coupled to a radioactive isotope, penetrate efficiently the target tissue, cross cell membranes and be retained long enough in vivo in the target tissue to effect the required dose delivery. DNA oligonucleotides may well have the necessary qualities to fill the requirements needed to become an efficient radioactivity vector.

We have shown in our laboratory that DNA oligonucleotides, conjugated or not with cholesterol, can be efficiently delivered locally in a vessel wall in vivo, cross cell membranes, become incorporated intracellularly and be retained in vivo for periods extending to at least one week. We and others (Azrin M et al, 1997, Cathet. Cardiovasc. Diagn., 41(3):231) have shown that commercially available catheters such as the Dispatch catheter, (Scimed, Minn., USA) or the Infiltrator catheter (Interventional Technologies, CA, USA) can effect local drug delivery of oligonucleotides in the above-mentioned fashion in coronary arteries of animals and humans. Thus, if one could combine a radioisotope with a DNA oligonucleotide to transport the isotope to the target cells using a local drug delivery catheter as described above, the concept of intratissular radiotherapy could be realized. Also, if said radiolabelled oligonucleotide could be loaded onto a stent surface, it could eventually be delivered to the vascular tissue after stent deployment at target site.

### SUMMARY OF THE INVENTION

With the recent development of site-specific drug delivery for vascular disease, according to the present invention, beta irradiation of the angioplasty site through molecular radiotherapy by delivering phosphorus 32 locally through a labeled DNA carrier (such as an oligonucleotide) at the dilatation site appears feasible and prevents smooth muscle cell proliferation and restenosis.

The present invention may be combined with other therapeutic modalities that have been shown efficient in arteries such as stenting. This local drug delivery strategy based on the use of the invention presented here may be applicable to all vascular proliferative disorders such as coronary and peripheral arterial restenosis, by-pass graft restenosis, arterio venous fistulas, etc. and cancer and metastasis therapy.

It is therefore an object of the present invention to provide a new and innovative approach to prevent restenosis and potentially any other vascular proliferative disease or/and treat other proliferative processes such as cancer and related metastasis.

It is another object of the present invention to provide DNA sequences that carry one or several radioisotopes, located internally within the DNA sequence, and which are able to prevent cell proliferation in vitro and, following local drug delivery to prevent the occurrence of restenosis in vivo.

Other objects of the invention will appear as the description follows.

In accordance with the present invention, there is provided a therapeutic substance comprising a radiolabeled DNA carrier (such as an oligonucleotide), wherein the radioisotope is located internally within the DNA sequence.

In accordance with the present invention, the DNA carrier act as a vector for radioisotope so that the radioisotope may penetrate the cell membrane and be retained intracellularly for a time sufficient for the radioisotope to effect an efficient dose therapy. The length and the specificity or affinity of the DNA carrier is not relevant. DNA carrier of up to 2000 bp in length, without specificity are shown to be effective for preventing restenosis. Preferably, thus the oligonucleotides are from 3 to 2000 bp in length, more preferably, the oligonucleotides can be of the size ranging from 3 to 500, 3 to 200, 3 to 100, or 10 to 100 in length

The radioisotope used to radiolabel the carrier (such as an oligonucleotide) of the present invention may be an alpha, beta or gamma emitter. Preferred radioisotopes in accordance with the present invention include, without limitation, ³²P, ³³P, ¹²⁵I, ¹³¹I, ³⁵S, ¹⁹⁸AU, ⁹⁰Y, ⁸⁹SR, ¹⁸⁶Re, ⁴⁵Ca and ¹⁵³Sm among others. The half-life of the preferred radioisotopes used in accordance with the present invention should vary between 10 hours and 1000 days.

The oligonucleotide carrier on which the radioisotope will be attached is a double-stranded DNA sequence, a single-stranded DNA sequence or DNA analog sequences thereof. The individual nucleotides can be chemically modified as follows.

1. The inter-nucleotide bond such as phosphodiester, phosphorothioate, methylphosphonate or any other covalent bond that can link individual nucleotides.

2. The hydrogen (H) normally located at 2′-position of the nucleotides can be substituted by other chemical moieties such as 2′-O-methyl, 2′-O-propyl, 2′-Fluoro, 2′-O-methoxyethyl that will confer an enhanced metabolic stability.

3. The purine and pyrimidine bases can be also chemically altered for the same reasons as mentioned in 2 above.

In accordance with a preferred embodiment, the oligonucleotide is conjugated with an antibody for cell-specific delivery of the oligonucleotide.

In accordance with another preferred embodiment, the oligonucleotide is conjugated to a hydrophobic moiety, such as cholesterol, to favorably influence its pharmacokinetic properties.

The radiolabeled DNA carrier of the present invention may be encapsulated in a liposomal formulation prior to its administration to the patient.

The radiolabeled carrier (e.g. oligonucleotide) of the present invention may be directly or indirectly attached to a stent surface to prevent uncontrolled cellular proliferation occurring in cases of restenosis.

The DNA carrier sequence may be a plasmid, such as a circular plasmid of viral or bacterial origin, in its complete or incomplete form. Such a viral plasmid used in accordance with the present invention may be adenovirus.

In accordance with another preferred embodiment, the oligonucleotide carrier sequence can either target a specific gene by any antisense mechanism or it can be an unrelated sequence. Any sequence of DNA of at least 2 nucleotides to about 5000 nucleotides may be used as a DNA or DNA analog oligonucleotide in accordance with the present invention, preferably a DNA sequence of 25 nucleotides or less. For example, the following sequences may be used but not restricted to:

| | | |
|---|---|---|
| cmyc | CAC GTT GAG GGG CAT | (SEQ ID NO:1) |
| cmyc sense | ATG CCC CTC AAC GTG | (SEQ ID NO:2) |
| FOS (sense | GCC CGA GAA CAT CAT | (SEQ ID NO:3) |
| and antisense) | | |
| Jun (sense | CCT CGC AGT TTC CAT | (SEQ ID NO:4) |
| and antisense) | | |
| | AAA AAA AAA AAA AAA TTT | (SEQ ID NO:8) |
| | TTT TTT TTT TTT TTT AAA | (SEQ ID NO:9) |
| | CCC CCC CCC CCC CCC GGG | (SEQ ID NO:10) |
| | CC GCG ACG ATG CCC CTC AAC GTT ACC ATC ACC | (SEQ ID NO:11) |

wherein the radioisotope may be located at any internal position in the sequence.

It is not a requirement for the current invention, although not a contraindication, to be an antisense sequence to a specific target. A similar and significant level of smooth muscle cell proliferation inhibition can be obtained in vitro by using oligonucleotides of either the sense or antisense sequence to the c-myc mRNA sequence. Both sense and antisense-labeled sequences result in the same level of proliferation inhibition since the therapeutic element of the molecule was the beta-emission from the radioisotope (Phosphorus 32) incorporated within the transfected sequence. Further tests have also revealed that any sequence without specificity or affinity, such as homonucleotides A, C, G or T, or any other sequence, when labeled internally will prevent smooth muscle cells proliferation.

The invention also relates to a method for preparing a radiolabeled DNA carrier sequence (such as an oligonucleotide) wherein the radioisotope is located internally within the DNA sequence, which comprises the steps of:

a) synthesizing a DNA sequence in at least two parts;

b) labeling the 5′ end of one of the two parts with a radioisotope;

c) hybridizing the two or more parts of step b) with a sequence capable of hybridizing under stringent conditions; and

d) ligating together the hybridized two or more parts to form a radiolabeled double-stranded DNA oligonucleotide.

In order to obtain a radiolabeled single-stranded oligonucleotide of the present invention, the method further includes a step e) after above step d), which comprises

e) separating the hybridized DNA and recovering the radiolabeled single-stranded DNA oligonucleotide sequence.

Also, when a double-stranded oligonucleotide having both strand radiolabeled is desired, the method further includes a step f) after above step e), which comprises

f) hybridizing together complementary radiolabeled single-stranded DNA oligonucleotides of step e).

When the two parts of step a) form an antisense sequence, the sequence capable of hybridizing of step c) is a corresponding sense sequence.

When the two parts of step a) form a sense sequence, the sequence capable of hybridizing of step c) is a corresponding antisense sequence.

The invention further relates to a method for the prevention of uncontrolled cell proliferation in a mammal, which comprises delivering a therapeutic substance as defined above to the mammal in situ where the uncontrolled cell proliferation takes place. For example, when the uncontrolled cell proliferation is a restenosis following angioplasty, therapeutic substance is delivered by site-specific intravascular delivery such as those described previously.

Another possibility is when the uncontrolled proliferation is cancer or a malignant tumor. For this case, the therapeutic substance may be coupled to an antibody or a peptide moiety. Such a peptide moiety include, without limitation, Transforming Growth Factor α (TGFα), TGFβ, cytokines and any growth factors. The coupled radiolabeled oligonucleotide may be given locally, in a site-specific manner, or systemically.

This therapeutic, internally radiolabeled oligonucleotide may be conjugated to other moieties, such as cholesterol, oleic acid or linoleic acid, to favorably influence its vascular pharmacokinetic properties. It may also be conjugated with an antibody to increase its cell specificity. The DNA sequence, which solely acts as a radioisotope carrier may be a sense or antisense sequence to a known genetic target. The sequence used does not represent the therapeutic portion of the molecule. The anti-proliferative activity of the molecule stems from the radioactive isotope attached to the DNA.

### BRIEF DESCRIPTION OF THE DRAWINGS

FIG. 1 illustrates a histological section of a rabbit carotid artery transfected by a transluminal approach with 80 micromolar of a fluorescein-labeled 15 mer DNA oligonucleotide;

FIG. 2 illustrates the retention of 15 mer DNA oligonucleotides in the vessel wall following local in vivo transfection.

FIG. 3 is a schematic representation of a ³²P-labeled DNA oligonucleotide sequence in accordance with one embodiment of the present invention;

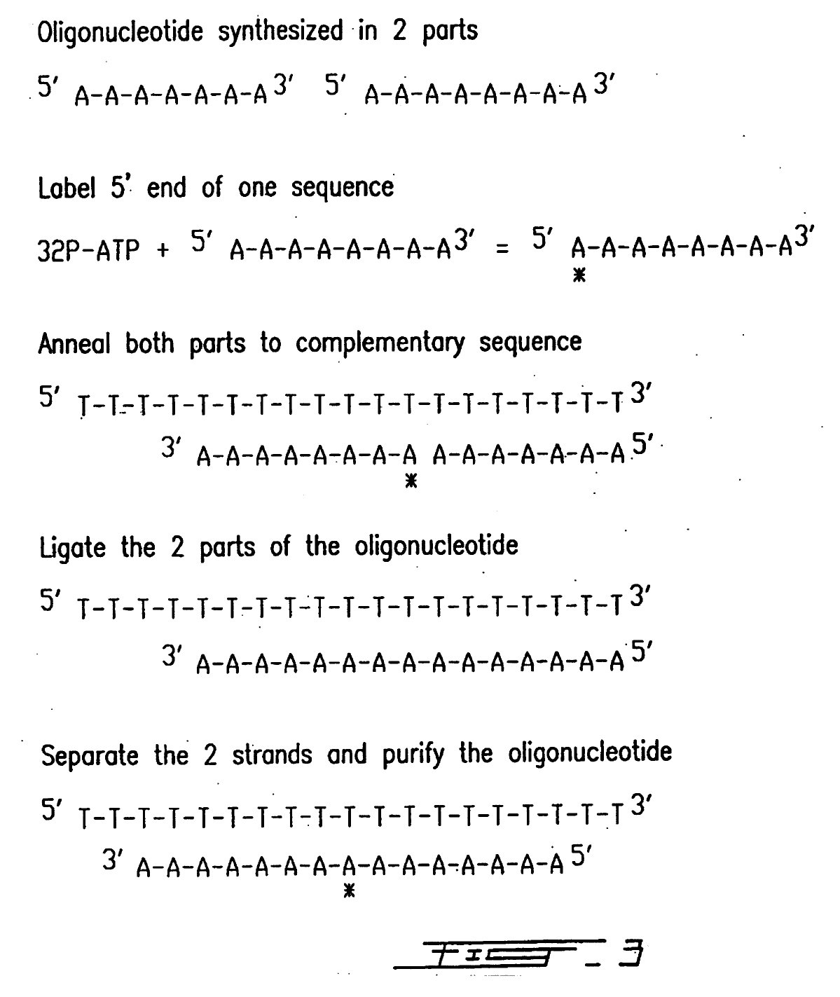

FIG. 4 shows the stability of a ³²P-labeled oligonucleotide (SEQ ID NO:1) following exposure to endonucleases and exonucleases in vitro;

FIG. 5 shows the dose-response curve following the addition of a ³²P-labeled oligonucleotide (SEQ ID NO:2) to smooth muscle cells in vitro;

FIG. 6 shows that sense and antisense sequences of ³²P labeled oligonucleotides have the same inhibitory potential on smooth muscle cell proliferation;

FIG. 7 shows the significant superiority of the labeled oligonucleotide when compared to unlabeled oligomers in the prevention of smooth muscle cell proliferation;

FIG. 8 shows a histological section of a control porcine coronary artery in which a stent was deployed;

FIG. 9 shows a histological section of a treated porcine artery in which a stent was deployed, followed by the local delivery of 80 micromolar of a 32 Phosphorus-labeled oligonucleotide (15 mer, SEQ ID NO:1);

FIG. 10 shows a bar graph illustrating the effect of various internally labeled oligonucleotides of the present invention on cell proliferation;

FIG. 11 illustrates morphometric analysis parameters such as the maximal intimal thickness (MIT) as determined by a radial line from the lumen to the external lamina at the greatest intimal thickening, the arc length of the medial fracture, traced through the neointima from one dissected medial end to the other, used to evaluate the extent of injury (fracture length), and area measurements of intima and media (in mm²);

FIGS. 12A to 12C represent photomicrographs of porcine coronary arteries in non-injured (FIG. 12A), balloon-injured (FIG. 12B) and ³²P-oligonucleotide-treated injured (FIG. 12C, 15 Gy) conditions; and

FIG. 13 represents histomorphometric analyses of control and ³²P-oligonucleotide (15 Gy)-treated coronaries.

### DETAILED DESCRIPTION OF THE INVENTION

Most of the approaches with ³²P described previously are related to a source remaining external to the target cells, are already patented, and several, namely the stent-based approach, would be very difficult to implement in daily practice for practical reasons. Applicants have gained an important expertise in the field of local drug delivery. Two technologies, that of a beta-emitting source and DNA carrier or oligonucleotide, to effect the prevention of restenosis, were combined in a unique and original way.

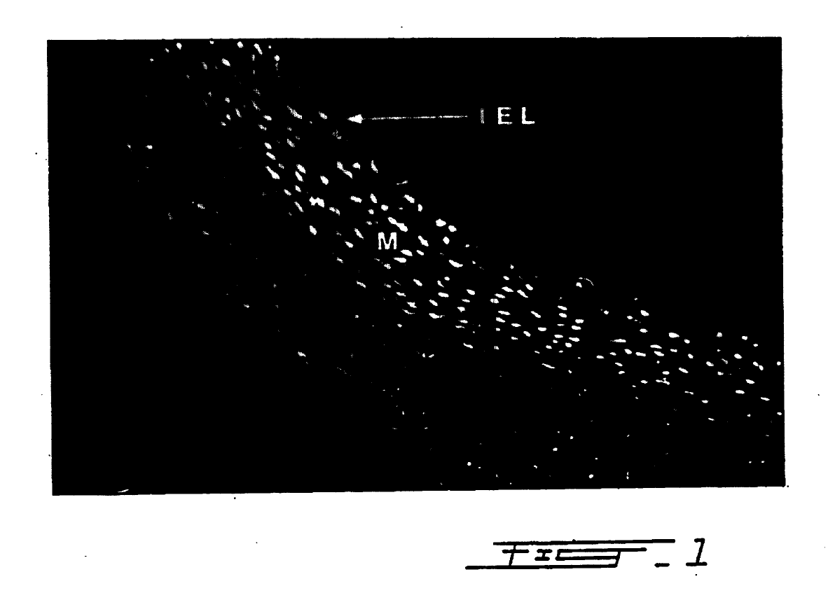

FIG. 1 shows a rabbit carotid artery transfected with 80 micromolar of DNA oligonucleotide (15 mer, phosphorothioate) over a period of 30 minutes. More than 90% of cells are transfected by the oligonucleotide with preferential nuclear localization, thus demonstrating the ability of such oligomers to penetrate the vascular tissue following local transfer and cross cell membrane, in vivo. IEL represents the internal elastic lamina and M represents the media of the vessel. Over 90% of cells comprised in the media of the artery were successfully transduced by the fluorescein-labeled oligonucleotide.

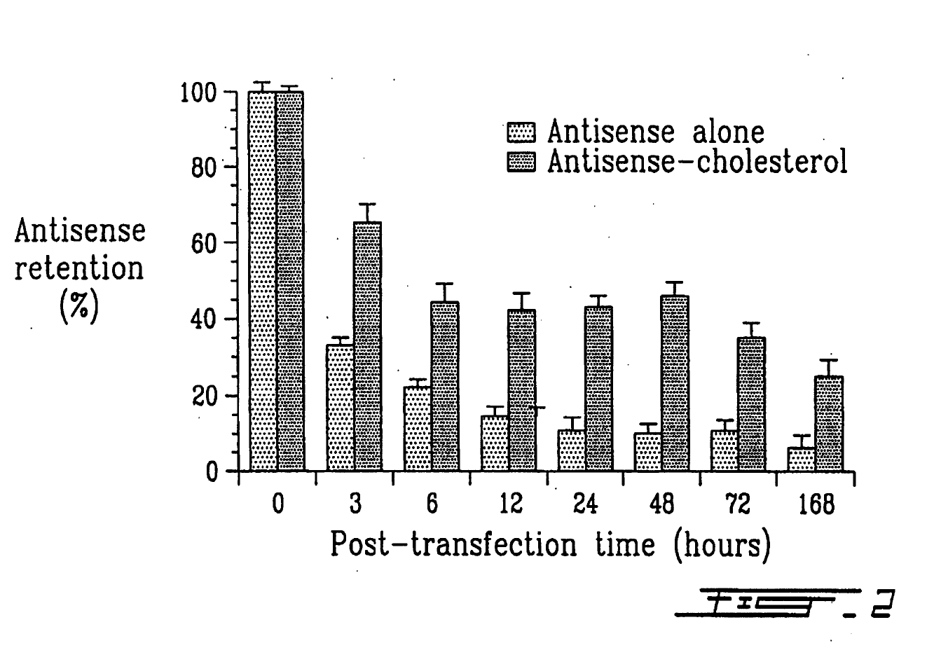

FIG. 2 shows that following local transfection in the rabbit carotid artery, the transfected DNA oligomer can remain in the vessel wall for periods extending over 1 week. Transfer was done with either cholesterol-conjugated or non-conjugated oligonucleotides. The short DNA sequences are retained for up to one week in the treated vessel site in vivo.

The preferred radioisotope in accordance with the present is ³²P, which is a pure beta emitter with an average energy of 0.69 MeV, a maximum energy of 1.71 MeV (maximum range of ˜8 mm in soft tissues) and a half life of 14.3 days. Using an efficient tissue carrier for ³²P has enabled to effect the radiotherapy from within the vessel wall and potentially within cells.

In accordance with the present invention, 15 mer oligonucleotides with sense or antisense sequence to the proto-oncogene c-myc were used to demonstrate the potential of a radiolabeled (³²P-labeled) oligonucleotide to inhibit proliferation of cells.

End labeling of oligonucleotides with a radioisotope (³²p) is a very common reaction in molecular biology. However, this end labeling is more difficult to achieve with phosphorothioate as compared to phosphodiester oligonucleotides. Furthermore, phosphodiester oligonucleotides are readily degraded in vivo (within hours) by nucleases after transfection. The labeling of the 5′ end or the 3 ′end of an oligonucleotide does not exhibit a strong stability and the label could be cleaved from the oligonucleotide once it is incubated with cells. A method to label the oligonucleotide in an internal position was used. The schematic representation of the method of preparing such a radiolabeled oligonucleotide is outlined in FIG. 1.

The sequence of the final product used to perform this demonstration is: CACGTTGA(*)GGGGCAT (SEQ ID NO:1) (the * indicates the position of the radioactive phosphorus atom). To achieve this result, the following three (3) different oligonucleotides of Table 1 below were used.

TABLE 1

| Oligonucleotide | Sequence | Characteristic | SEQ ID NO |
|---|---|---|---|
| 1. c-mycl9 | ATGCCCCTCAACGTGAAAA | phosphorothioate | SEQ ID NO:5 |
| | | or phosphodiester | |
| 2. c-myc1 | CACGTTGA | phosphorothioate | SEQ ID NO:6 |
| 3. c-myc2 | GGGGCAT | phosphorothioate | SEQ ID NO:7 |
| | | phosphodiester | |

The third oligonucleotide (c-myc2) is a mixed phosphorothioate-phosphodiester molecule. The first 2 internucleotide bounds are phosphodiester while the remaining bounds are phosphorothioates. The synthesis of the internally labeled oligonucleotide involves the 5′ end labeling of c-myc2 followed by the annealing of c-myc2 and c-myc1 to c-myc19, then by the ligation of c-myc2 to c-myc1, and finally by the separation of c-myc2-c-myc1 from c-myc19.

The first reaction is the labeling of the c-myc2 oligonucleotide at the 5′ end. The labeling is achieved by incubating between 50 and 100 pmole of the oligonucleotide with 50 μCi of g ³²P-ATP and 2 units of T4 polynucleotide kinase at 37° C. for 2×30 minutes (a second input of the kinase is made after the first 30 minutes). The unincorporated ³²P is removed from the mix by gel filtration.

For the annealing the ³²P labeled c-myc2 is recovered and incubated with equimolar quantities of c-myc19 and c-myc1 for 30 minutes at 55° C. in presence of 12.5 mM TRIS-HCl (pH 8.5), 12.5 mM MgCl₂, and then cooled to room temperature.

The ligation of c-myc2 to c-myc1 is done by incubating the annealing mix overnight at 16° C. in the presence of 33 mM CH₃COOK, 1 mM ATP, and 14 units of T4 DNA ligase.

To separate the ligated c-myc2-c-myc1 from the c-myc19, an equal volume of formamide buffer (80% formamide, 10 mM EDTA, 1 mg/ml bromophenol blue, xylene cyanol) is added to the ligation mix, the sample is then heated 5 minutes at 65° C. and then loaded on a 20% polyacrylamide-urea gel for electrophoresis. After migration, the band corresponding to the ligated c-myc2-c-myc1 (15 bases long) is cut from the gel. The cut piece of gel is crushed in a fine powder and the powder is incubated with 2 volumes of TE (10 mM TRIS-HCl (pH 7.5), 1 mM EDTA) for 30 minutes at 55° C. The eluate is recovered after centrifugation and desalting is achieved by affinity chromatography. Using this method it was possible to produce an internally ³²P-labeled oligonucleotide. The activity of the recovered oligonucleotide was of the order of 1.2 μCi.

The synthesis of all oligonucleotides is carried out in an oligonucleotide synthesizer sold by Applied Biosystems under the designation 392 DNA/RNA Synthesizer™. After synthesis, the oligonucleotides are purified in Poly-Pak™ II columns bought from Glen Research. Alternatively, they can be purified by HPLC (High Pressure Liquid Chromatography).

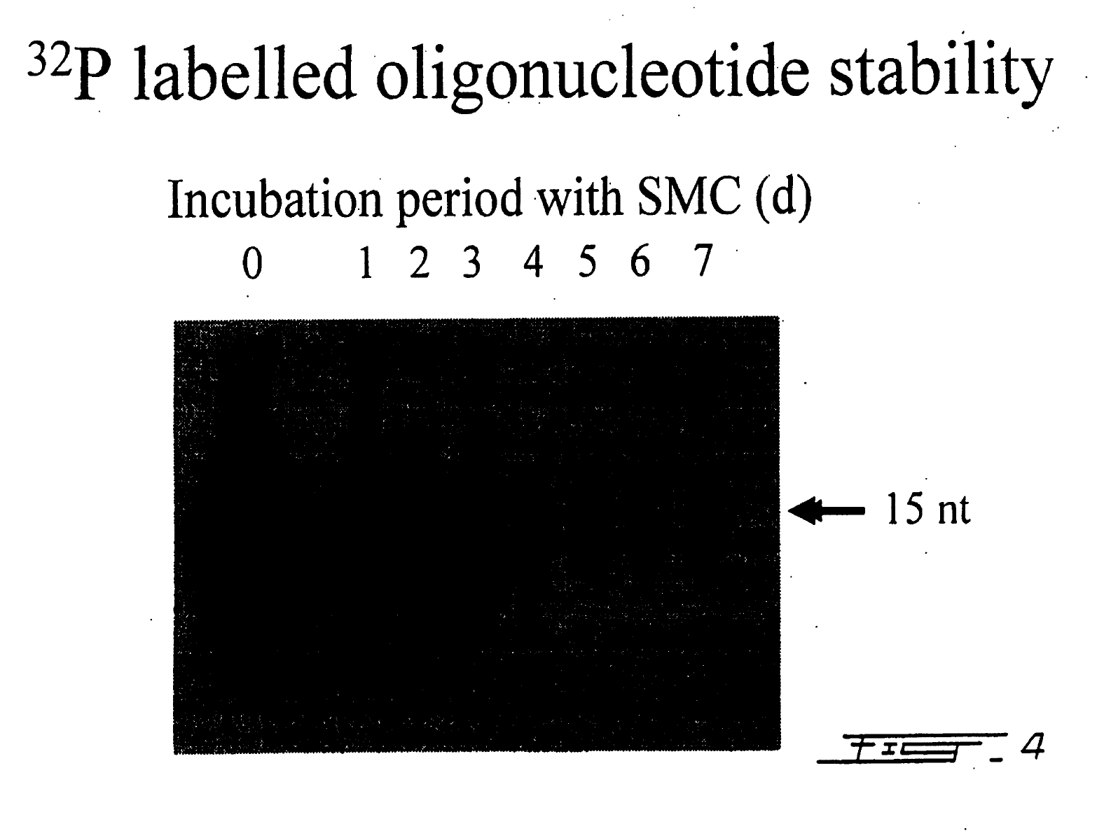

The effect of internally ³²P-labeled oligonucleotides was verified on smooth muscle cell growth. To measure proliferation of smooth muscle cell the tritiated thymidine incorporation assay was used. The cells are made quiescent by incubating them in a starvation medium. Proliferation is activated by increasing the bovine fetal serum content of the medium. The labeled oligonucleotide was added to the cells in quadruplet wells at the time of the activation. Tritiated thymidine was added to medium 12 hours after the activation and determination of the incorporation of the tritiated thymidine by the cells was determined after a further incubation of 12 hours. Stability of the ³²P-labeled oligonucleotide was also assessed by incubating a dose of 0.2 μCi of the oligonucleotide in the presence of smooth muscle cells. After an incubation of 7 days, the integrity of the ³²P-labeled oligonucleotide was confirmed by polyacrylamide-urea gel electrophoresis (FIG. 4). Stability of the molecule is shown for a period of at least 7 days.

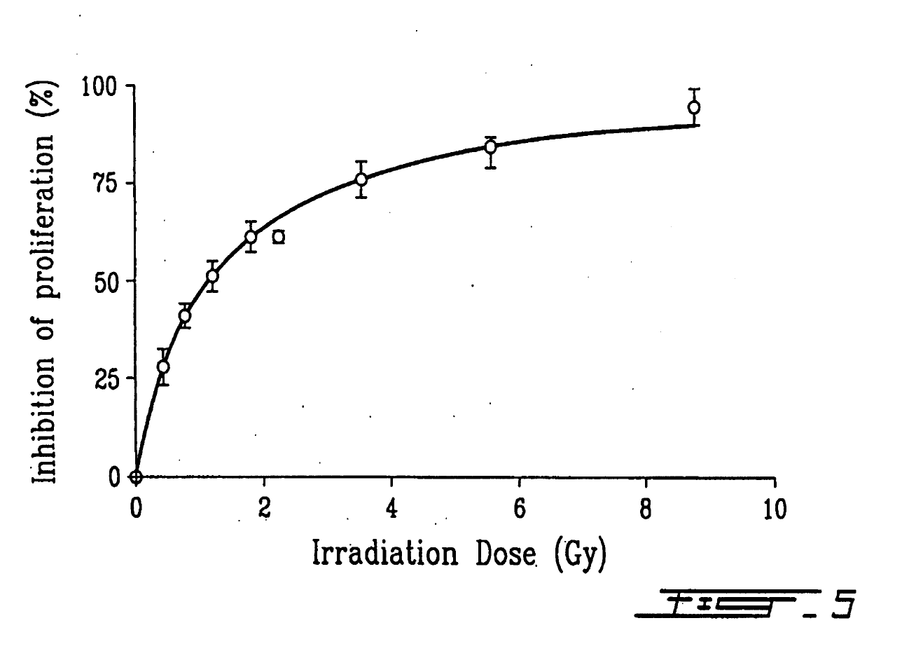

A dose-response curve of the inhibition of porcine and human smooth muscle cell proliferation was obtained using the above-described method (FIG. 5). The experiments were done by adding a ³²P-labeled oligonucleotide (SEQ ID NO:2) to smooth muscle cells grown in vitro.

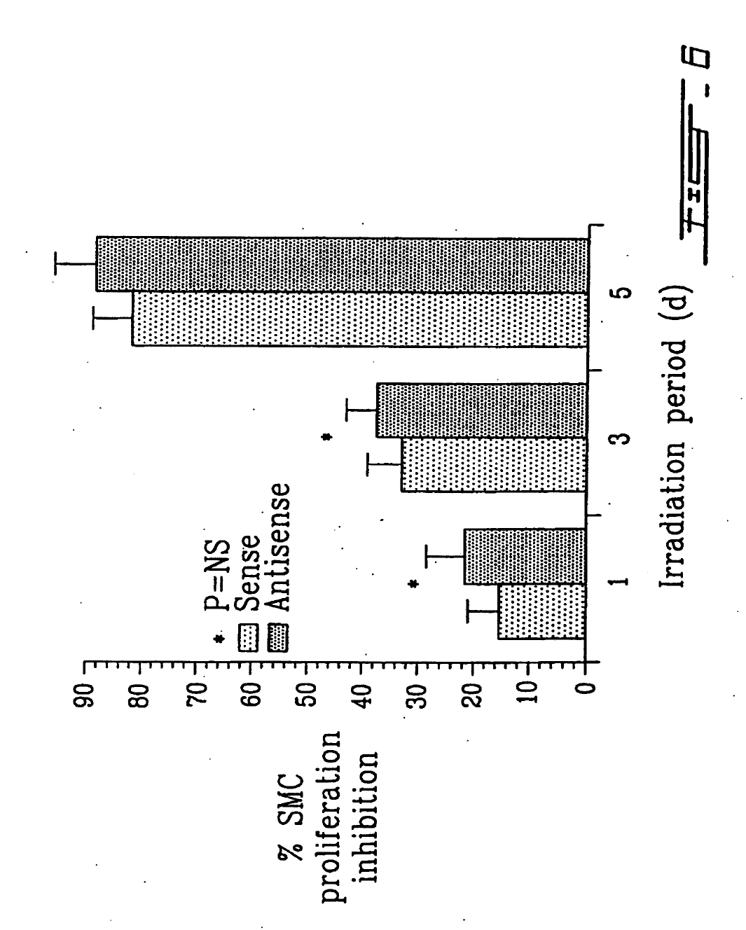

The inhibitory effects of beta-irradiation on the cell proliferation index was represented as the relative percentage of proliferation obtained in the treated cells compared to that in the control, non-irradiated cells. The effect of the unlabeled oligonucleotide (4, 8 and 20 nmol/L, SEQ ID NO:2, sense to c-myc) on cell proliferation was estimated to be less than 10%. A significant inhibition of human and porcine smooth muscle cell proliferation, however, was obtained with the 32 phosphorus-labeled oligonucleotide (SEQ ID NO:2) in a dose dependant manner (0.4 to 8.7 Gy). This inhibition was seen with both sense and antisense sequences (FIG. 6) underlying the fact that this invention is based on the therapeutic properties of the radioisotope carried by the DNA vector and not the sequence specificity of the oligonucleotide. This is explained by the fact that the therapeutic component of the molecule stems from the radioactivity emitted from the radioisotope inserted within the DNA sequence.

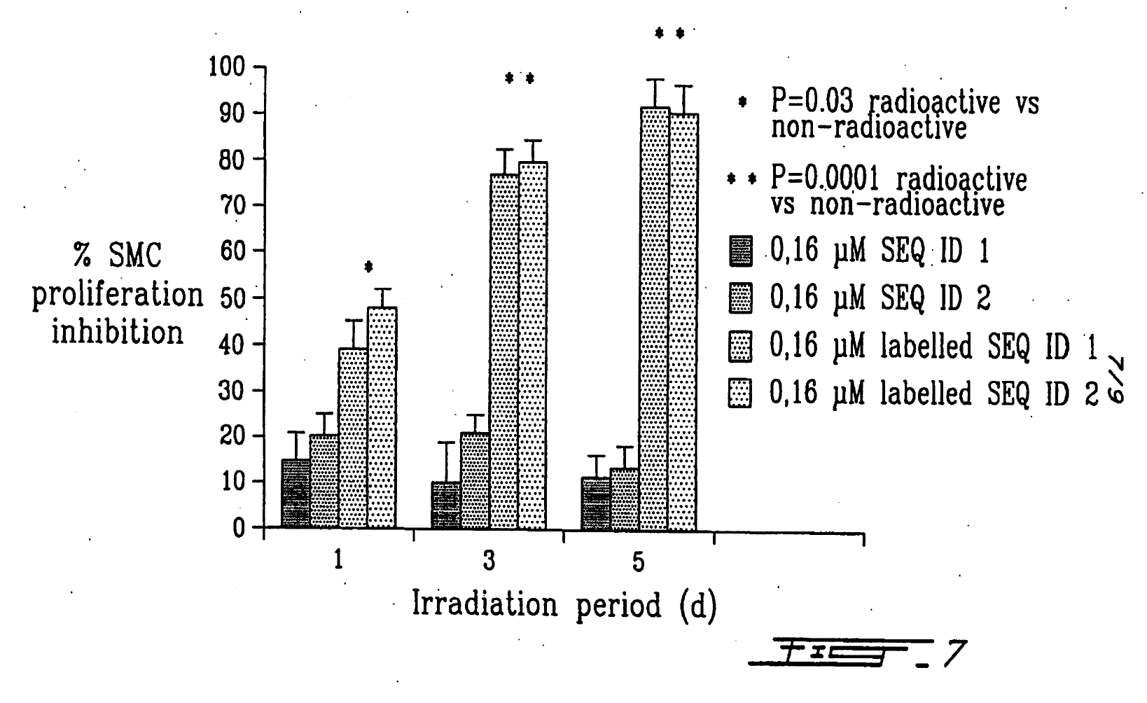

³²P-labeled oligonucleotides were much more efficient in the inhibition of smooth muscle cell proliferation than unlabeled DNA as shown in FIG. 7. These results demonstrate the marked advantage of treating cells with the labeled DNA vector.

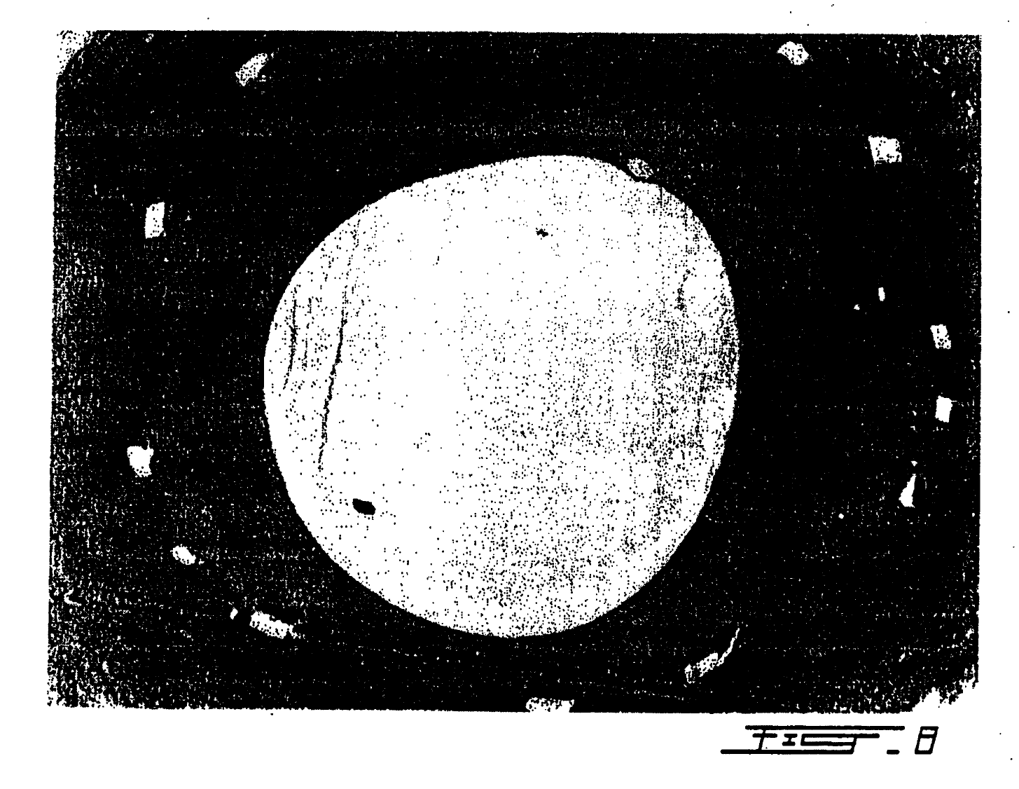

In order to demonstrate the efficiency of ³²P-labeled oligonucleotides (SEQ ID NO:2) to prevent smooth muscle cell proliferation in vivo, we examined the efficiency of these agents in a porcine coronary artery restenosis model. The restenotic lesion was created by the overstreching of a coronary stent in a porcine coronary artery. The resulting response to injury induces the proliferation of smooth muscle cells to the neointima and a reduction of the luminal area. In a control artery, the stent was deployed with a ratio of stent to artery of 1.3/1, using conventional angioplasty balloon catheters. No further treatment were given to the animal. Four weeks later, the animal was sacrificed and the stented artery processed for histological analysis. As shown in FIG. 8, an intense reaction to the stent overstrech is seen with the formation of an important neointimal layer that reduces significantly the luminal area. Notice the severe proliferative response obtained at 28 days forming an important neointimal layer that reduces significantly the luminal area.

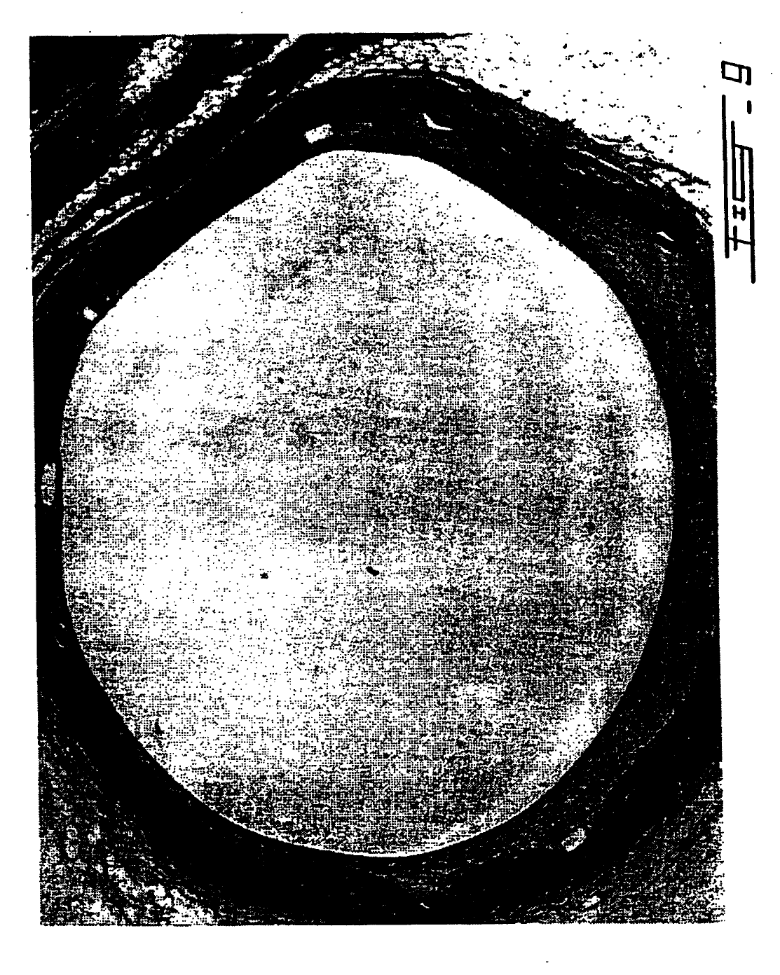

In contrast, a porcine coronary artery stented in the same fashion but also treated by the local administration of 2 microcuries of ³²P-labeled oligonucleotides (SEQ ID NO:2) showed a significant reduction, over 85% reduction (FIG. 9) in the neointimal hyperplasia usually seen following stent deployment (FIG. 8). At 28 days following stent implantation and local delivery of the radioactive oligonucleotide, there is a significant reduction in neointimal layer formation as compared to that seen in control arteries (FIG. 7). The oligonucleotide was delivered in the porcine coronary artery prior to the stent positioning with the Dispatch catheter. Local delivery was done in 15 minutes.

Phosphorothioate oligonucleotides have proved to be very good candidates to achieve intratissular vector for radiotherapy since their stability after transfection has been demonstrated for a period of at least 7 days. The results confirmed the feasibility of this approach. In vitro data shows that:

radiolabeled (³²P) oligonucleotides achieve a dose-dependent inhibition of smooth muscle cell proliferation;

this inhibition level often reaches 100% with a dose of 8.7 Gy mediated by the labeled DNA;

high efficiency inhibition is not dependent on the sequence of the DNA but on the radioactivity level given to the cells;

the length of the labeled DNA does not appear to significantly influence the level of smooth muscle cells proliferation inhibition.

the level of inhibition of smooth muscle cell proliferation obtained with radiolabeled (³²P) oligonucleotide is greatly superior to that obtained with non radioactive identical DNA sequences, for the same amount of DNA used; and

a maximal inhibition level has been attained with radiolabeled (³²P) oligonucleotide (100% inhibition at 8.7Gy) whereas this inhibition level has never been observed with conventional oligonucleotides.

Based on these data and the demonstration of the feasibility to locally deliver the radiolabeled (³²P) oligonucleotides at the site of angioplasty, it is reasonable to conclude that in vivo prevention of smooth muscle cell proliferation is expected following local delivery of radiolabeled (³²P) oligonucleotides. This local, site-specific, delivery can be performed in coronary and peripheral arteries by using local drug delivery catheters that are commercially available, such as the “Dispatch”, “Transport” catheter or “Infiltrator” catheter.

Also based on the data presented in the present application, it is reasonable to conclude that oligonucleotides as short as 3 bp and up to 2000 bp (such as for plasmid) are contemplated. The Applicants have demonstrated that the effectiveness of the present invention is not length-dependent or affinity-dependent of the oligonucleotides.

The above method of radiolabeling DNA carrier or oligonucleotides with ³²P in such a way to prevent its degradation by nuclease enzymes can also be applied to the field of oncology, thereby providing a mean for the specific delivery of therapeutically effective radiation dose to the tumor while minimizing normal tissue exposure.

This can be achieved for example by first radiolabeling a DNA carrier sequence or oligonucleotide that has an high affinity with cancer cells genetic material. Then the radiolabeled DNA or oligonucleotide sequence is bonded to a monoclonal antibody (MoAb) which recognizes tumor associated antigens. Such a bond between the radiolabeled DNA sequence and the MoAb can be done through an aminolink with the use of “Peptide Nucleic Acids” (PNA's) specifically designed for that purpose.

### EXAMPLE I

### Effect of ³²P internally labeled oligonucleotides on smooth muscle cell proliferation

The effect of a ³²P internally radiolabeled oligonucleotide has been demonstrated hereinbefore on proliferation of smooth muscle cells in vitro. The oligonucleotide of the present invention was of a definite length and composition. The present example extends the principle of the invention mentioned above. The principle of the cell proliferation inhibition is the same as above, mainly the effect of radiation provided by the radioisotope used to radio-label internally the oligonucleotide. The oligonucleotide serves only as a carrier or transporter for the radioactive element. To demonstrate that the effect of the radiation does not change with the modification of the oligonucleotide, several oligonucleotides that were internally labeled with ³²p were designed and their effect on smooth muscle cells proliferation was verified. The following homo-oligonucleotide sequences of different length have been used:

| | | |
|---|---|---|
| Homo-oligonucleotide A | AAAAAAAAAA AAAAATTT | (SEQ ID NO:8) |
| Homo-oligonucleotide T | TTTTTTTTTT TTTTTAAA | (SEQ ID NO:9) |
| Homo-oligonucleotide C | CCCCCCCCCC CCCCCGGG | (SEQ ID NO:10) |
| 32-mers oligonucleotide | CCGCGACGAT GCCCCTCAAC GTTACCATCA CC | (SEQ ID NO:11) |

For SEQ ID NO:8, ³²P labeling is done at the 5′ end of SEQ ID NO:12 (5′ AA AAA AAA TTT 3′). The labeled sequence is then ligated to SEQ ID NO:13 (5′ A AAA AA A 3′) using SEQ ID NO:14 (5′ AAA TTT TTT TTT TTT TTT CCC 3′) as a template. The final product gives SEQ ID NO:8, labeled on A at position 8. As a control, SEQ ID NO:8 was also synthesized entirely without the radioactive element.

For SEQ ID NO:9, ³²P labeling is done at the 5′ end of SEQ ID NO:15 (5′ TT TTT TTT AAA 3′). The labeled sequence is then ligated to SEQ ID NO:16 (5′ T TTT TTT 3′) using SEQ ID NO:17 (5′ TTT AAA AAA AAA AAA AAA CCC 3′) as a template. The final product gives SEQ ID NO:9, labeled on A at position 8. As a control, SEQ ID NO:9 was also synthesized entirely without the radioactive element.

For SEQ ID NO:10, ³²P labeling is done at the 5′ end of SEQ ID NO:18 (5′ CC CCC CCC GGG 3′). The labeled sequence is then ligated to SEQ ID NO:19 (5′ C CCC CCC 3′) using SEQ ID NO:20 (5′ CCC GGG GGG GGG GGG GGG AAA 3′) as a template. The final product gives SEQ ID NO:10, labeled on A at position 8. As a control, SEQ ID NO:10 was also synthesized entirely without the radioactive element.

For SEQ ID NO:11, ³²P labeling is done at the 5′ end of SEQ ID NO:21 (5′ AC GTT ACC ATC ACC 3′). The labeled sequence is then ligated to SEQ ID NO:22 (5′ CCG CGA CGA TGC CCC TCA 3′) using SEQ ID NO:23 (5′ GGT GAT GGT AAC GTT GAG GGG CAT CGT CGC GGA AA 3′) as a template. The final product gives SEQ ID NO:11, labeled on A at position 19. As a control, SEQ ID NO:11 was also synthesized entirely without the radioactive element.

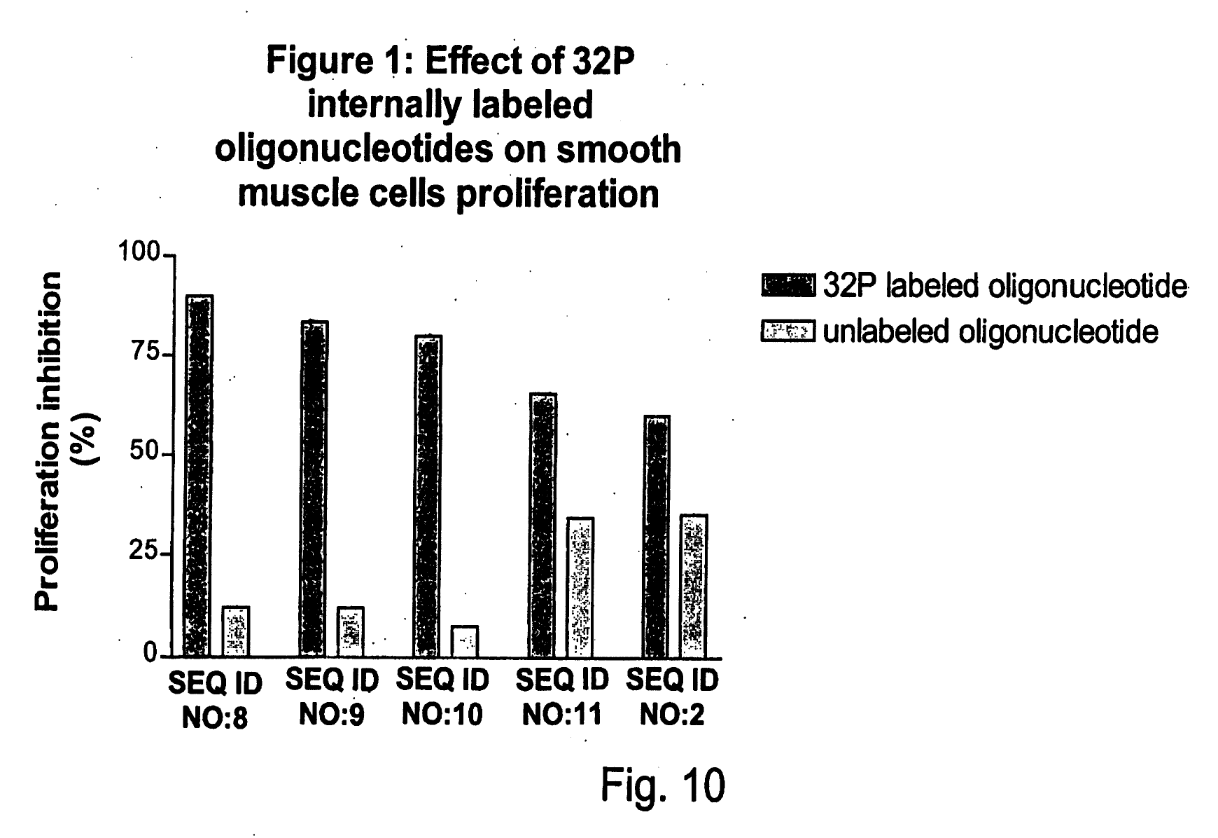

The internally labeled oligonucleotides described herein were used in presence of cultured human smooth muscle and their effect on cell proliferation was assessed by the thymidine incorporation index. The results are presented in FIG. 10.

Alternatively, an oligonucleotide having two ³²P internally within has been synthesised. This oligonucleotide has the following sequence:

5′CCGCGACGA*T GCCCCTCAA*C GTTACCATCA CC (SEQ ID NO:24)

wherein the * represents the ³²P radioisotope.

### EXAMPLE II

### Prevention of neointima proliferation in balloon injured arteries

Animal Model and Experimental Procedures

Domestic pigs (30-40 kg, 2 to 3 months, Primiporc Farm Inc., St Gabriel de Brandon, Quebec, Canada) were acclimated for one week in a room, where temperature, humidity and light-dark cycle were controlled. They were fed with standard pig chow (Purina Hog Tech 15.5% Pig Grower®) and tap water ad libitum. Whenever requireds, the domestic pigs were anesthetized with thiopental sodium (iv) and maintained in this stage with a mix of isoflurane 2% and oxygen to undergo classic angiographic procedure. The angiography was performed in at least two near orthogonal views that visualize the target site of left circumflex artery (LCX) of the pig. An IntraVascular UltraSound (IVUS) measure was done to assess the vessel size for adequate injury and local drug delivery procedure.

Local Drug Delivery Procedure

The radioactive oligonucleotide of the present invention, more particularly a 15-mer single strand DNA molecule, was used as a molecular delivery mode of β-particles to target specifically vascular cells. The ³²P-oligonucleotide, highly stable in the presence of cell layers (Fareh J, et al., Circulation, 99 (11):1477-1484, 1999), was designed with phosphorothioate bonds to increase its physical stability. The Infiltrator® catheter (InterVentional Technologies, San Diego, Calif.) was used for a site-specific administration of the ³²P-oligonucleotide. The exact location of infiltrator® balloon was verified and recorded with an injection of contrast media. After proper positioning of the drug delivery device at the selected site, the balloon was inflated to 2-4 atmospheres. The adequate apposition of the needles within the vessel wall was verified with contrast media. A total bolus of 0.6 mL of ³²P-oligonucleotide, diluted in contrast media, was then slowly infused over 60-90 seconds, with continuous monitoring of ECG to assess any sign of ischemia. Following balloon deflation, control angiography was performed to document any residual luminal stenosis or vessel wall dissection. If spasm was documented, 1 mL of nitroglycerin solution at a concentration of 0.3 mg/mL was injected intra-coronary.

Balloon Injury Model

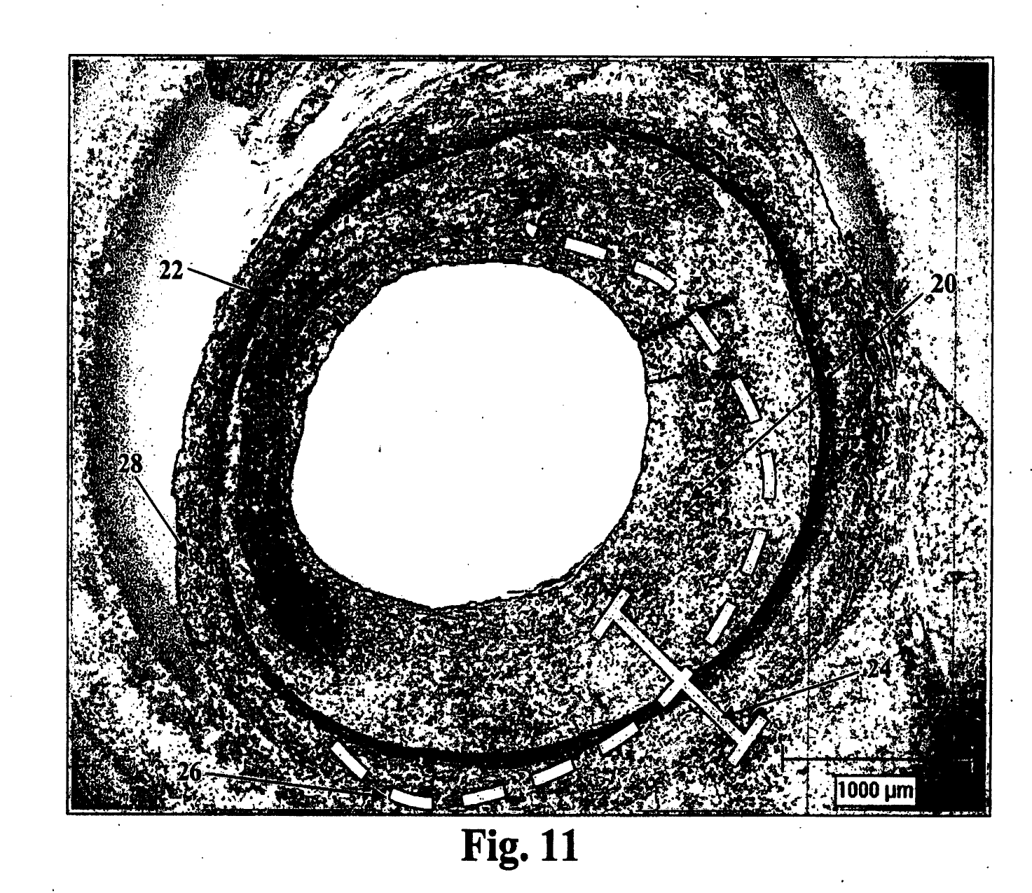

The overstretch injury model was used to induce a vessel wall injury resulting in measurable neointimal hyperplasia in porcine coronary arteries. For that issue, only LCX arteries were used. Following an IVUS measurement, coronary overstretch was applied (ratio 1.2:1.0) by three balloon inflations of 30 seconds, as previously described (Gravanis MB, et al., Cardiovasc. Pathol., 2(4):263-273, 1993; Waksman R, et al., Circulation, 91:1553-1559, 1995; and Wiedermann L G, et al., J. Am. Coll. Cardiol., 25:1451-1456, 1995). Under fluoroscopic guidance, infiltrator® catheter was then exactly localized at the dilated site. Two injured groups were used: one underwent balloon injury only (control group of n=8 animals) and one underwent vessel injury followed by administration of ³²P-oligonucleotide (n=8, 1.68 mCi/0.6 mL). Regarding the dosimetry estimation, the activity amount was chosen to reach a cumulative dose of 15 Gy at the media-adventitia (22 and 28, respectively on FIG. 11) interface for a 28-day follow-up. At the end of the procedure, control angiography was then performed to document any residual luminal stenosis or vessel wall dissection in both groups.

Histomorphometry of Porcine Coronary Arteries

At 28 days, pigs were euthanized by KCl injection to remove the heart for an in situ fixation in 10% neutral buffered formalin at 100 to 110 mmHg pressure for 40 minutes. Fixed heart were then sent to ClinTrials Bioresearch (Senneville, Quebec, Canada) for tissue processing (CTBR protocol number: #99492). Arteries were dissected out of the epicardium to be dehydrated using graded alcohol and xylene. Arteries were embedded in paraffin and three radial cross sections (5-7 μm) per artery were performed (proximal, medial and distal regions). Each segment was stained with Verhoeff van Gieson stain to be analyzed with a computer-assisted morphometric program (Clemex Biotechnologies Inc., Longueuil, Quebec, Canada). Computer-assisted morphometry was assessed by an experienced observer blinded for the coronary treatment. As illustrated in FIG. 11, area measurements of intima 20 and media 22 (in mm²) were obtained by tracing the perimeter of both internal and external elastic lamina as previously described (Waksman et al., supra; and Wiedermann et al., supra) . Neointimal hyperplasia was estimated by calculating the intima 20 to media 22 ratio (I/M) and by measuring the maximal neointimal thickness 24 (MIT, maximal narrowing of intima in mm) (Waksman et al., supra). Index injury was evaluated by measuring the perimeter of the fracture of the internal elastic lamina for each section (FIG. 11). Sections where no fracture was observed were not analyzed. Corrections for the vessel size was performed by measuring the residual lumen area in mm² (Waksmann et al., supra).

Statistical analysis

All data are expressed as mean ± SEM. Morphometric results were compared using the unpaired Student t test for parametric values or the Mann and Whitney rank test for non-parametric distribution. Differences were considered significant when P<0.05.

Vehicle

Various types of vehicle were used in vivo with the oligonucleotides of the present invention. The types of vehicle were based on hydrophilic (saline and heparin, Sigma), lipophilic (anhydrous alcohol-castor oil, Sigma), 1% cellulose (Sigma), 50% galactose (Sigma), levovist® (ultrasound contrast media, 99.9% galactose-0.1% palmitic acid, Berlex Canada Inc., Lachine, Quebec, Canada), 30% dextran, Hyskon® 32% (Hysteroscopy fluid, 32% dextran 70, Medisan Pharmaceuticals) and iodine formulations (radiopaque contrast media, Hexabrix-320®, Optiray 320® and MD-76®, Mallinckrodt Medical, Pointe Claire, Quebec, Canada).

Results

Following balloon injury or/and drug delivery procedure, no provisional stenting was done, as no dissection was observed in both groups.

Histomorphometric Analysis in Radioactive Coronary

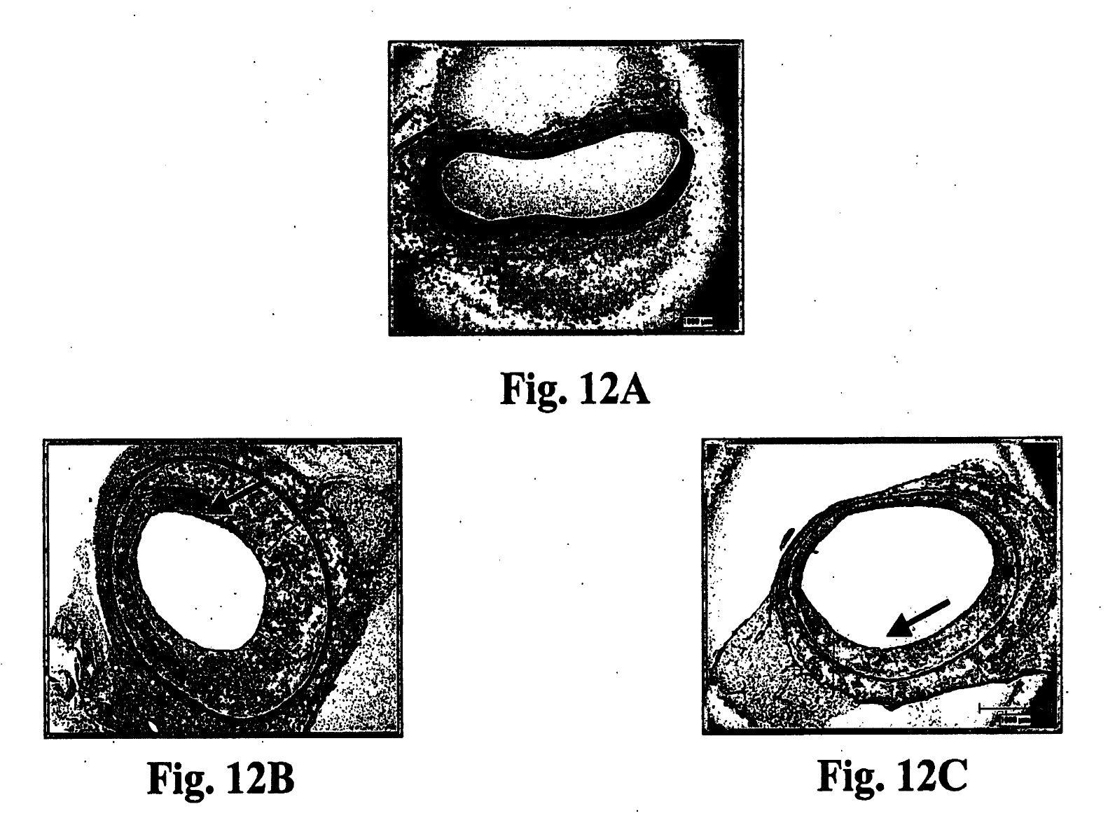

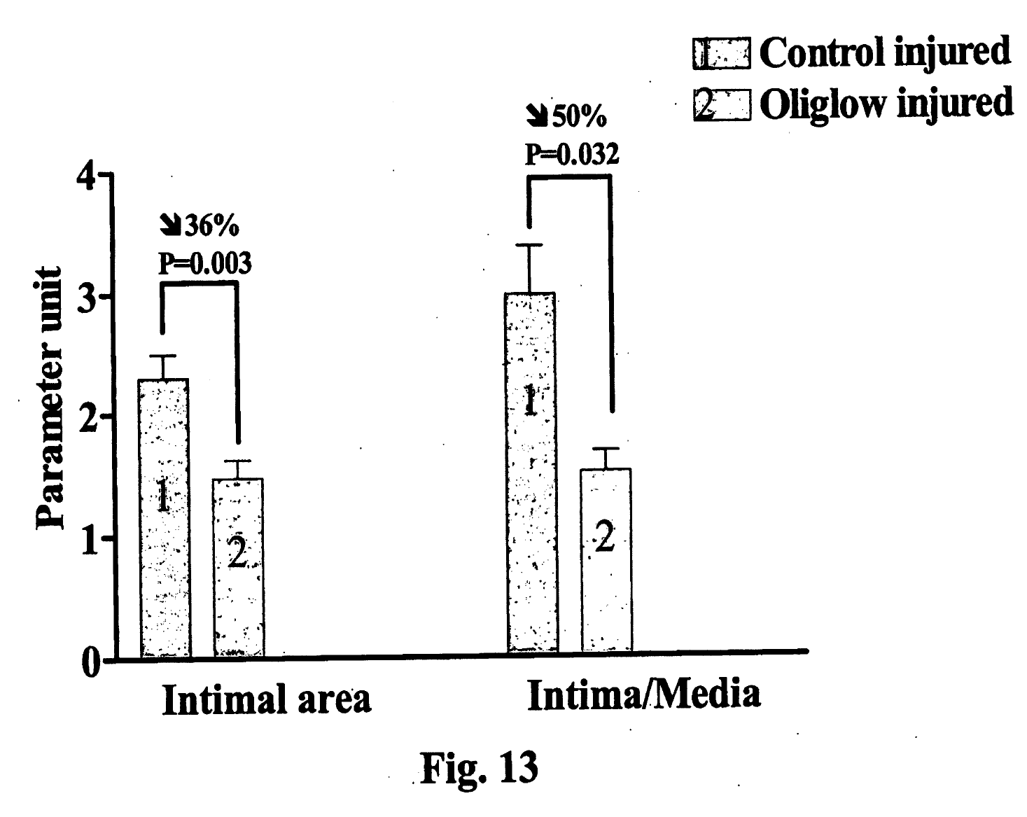

FIGS. 12A to 12C illustrate representative non-injured (FIG. 12A), balloon injured (FIG. 12B) and ³²P-oligonucleotide-treated injured arteries (FIG. 12C, 15 Gy). Following 28 days of initial injury, coronary arteries treated with the ³²P-oligonucleotide were characterized by a lower neointimal response when compared to control injured arteries (FIG. 12C). As reinforced by histomorphometric analysis (FIG. 13), intima area (1.47±0.15 vs 2.30±0.22 mm2 for control coronaries, P=0.003) and intima to media ratio (1.51±0.17 vs 2.98±0.46 for control coronaries, P=0.032) (FIGS. 12A to 12C), as well as MIT (0.6±0.05 vs 0.75±0.05 mm for control coronaries, P=0.038) were significantly reduced in ³²P-oligonucleotide-treated arteries, when compared to control injured arteries.

In parallel to that, residual lumen area was significantly higher in ³²P-oligonucleotide-treated arteries (0.55±0.03 vs 0.65±0.03 mm2 for control coronaries, P=0.027), whereas local administration of ³²P-oligonucleotide did not alter media area (1.1±0.06 vs 0.95±0.05 mm2 for control coronaries, P=0.09). Control and ³²P-oligonucleotide-treated coronaries underwent similar balloon injury as assessed by statistical equal level of fracture length 26 between both groups (2.3±0.2 vs 2.6±0.2 mm for control coronaries, P=0.516).

In FIG. 12B, balloon injury (B/A:balloon to artery ratio 1.1-1.2) induced disruption of internal elastic lamina (arrow), responsible of neointimal formation. Even the elastic disruption (arrow), Oliglow-treatment lowered neointimal proliferation. The bar represents a distance of 1000 μm.

In FIG. 13, bar graphs show the significant reduction of intima area (mm²) and intima to media ratio (I/M) in Oliglow-treated arteries. Data are expressed as mean ± SEM. N=8 per group. Student t test was used for statistical comparisons.

Conclusion

The results obtained in vivo clearly illustrate the effect of ³²P-oligonucleotide treatment in the prevention of reparative processes following balloon injury in porcine coronary arteries. Site-specific administration of ³²P-oligonucleotide, leading to a cumulative dose of 15 Gy, succeeds in reducing significantly neointimal proliferative response to balloon injury in swine.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. An anti-proliferative substance for preventing uncontrolled cellular proliferation, which comprises a radiolabeled DNA carrier, wherein a radioisotope is located internally within the DNA sequence, at 5′ end or at 3′ end, and wherein said radiolabeled DNA carrier penetrates cell membrane and is retained intracellularly for a time sufficient for the radioisotope to effect an efficient dose therapy.

2. The anti-proliferative substance according to claim 1, wherein said carrier is an oligonucleotide.

3. The anti-proliferative substance according to claim 2, wherein said oligonucleotide is linear.

4. The anti-proliferative substance according to claim 1, wherein said carrier is a plasmid.

5. The anti-proliferative substance according to claim 4, wherein said plasmid is circular.

6. The anti-proliferative substance according to claim 5, wherein said plasmid is of viral or bacterial origin.

7. The anti-proliferative substance according to claim 1, wherein said radioisotope is selected from the group consisting of ³²P, ³³P, ¹²⁵I, ¹³¹I, ³⁵S, ¹⁹⁸AU, ⁹⁰Y, ⁸⁹SR, ¹⁸⁶Re, ⁴⁵Ca and ¹⁵³Sm.

8. The anti-proliferative substance according to claim 3, wherein said oligonucleotide is a double-stranded DNA sequence or a single-stranded DNA sequence.

9. The anti-proliferative substance according to claim 3, wherein said oligonucleotide is conjugated with an antibody for cell-specific delivery.

10. The anti-proliferative substance according to claim 8, wherein said DNA oligonucleotide sequence is a single-stranded sense DNA sequence for hybridization to a specific genetic target.

11. The anti-proliferative substance according to claim 8, wherein said DNA oligonucleotide sequence is a single-stranded antisense DNA sequence for hybridization to a specific genetic target.

12. The anti-proliferative substance according to claim 1, which comprises DNA sequences of at least about 2 to about 2000 nucleotides.

13. The anti-proliferative substance according to claim 12, wherein the DNA sequence is selected from the group consisting of
(SEQ ID NO:1)
CAC GTT GAG GGG CAT
(SEQ ID NO:2)
ATG CCC CTC AAC GTG
(SEQ ID NO:3)
GCC CGA GAA CAT CAT
(SEQ ID NO:4)
CCT CGC AGT TTC CAT
(SEQ ID NO:8)
AAA AAA AAA AAA AAA TTT
(SEQ ID NO:9)
TTT TTT TTT TTT TTT AAA
(SEQ ID NO:10)
CCC CCC CCC CCC CCC GGG
(SEQ ID NO:11)
CC GCG ACG ATG CCC CTC AAC GTT ACC ATC ACC
wherein the radioisotope is located at any internal position in the sequence.

14. The anti-proliferative substance according to claim 3, wherein the oligonucleotide is conjugated to at least one selected from the group consisting of a stent surface, cholesterol, oleic acid, linoleic acid, TGFα, antibody, TGFβ, cytokines and growth factors.

15. The anti-proliferative substance according to claim 13, wherein the radioisotope is selected from the group consisting of ³²P, ³³P, ¹²⁵I, ¹³¹I, ³⁵S, ¹⁹⁸AU, ⁹⁰Y, ⁸⁹SR, ¹⁸⁶Re, ⁴⁵Ca and ¹⁵³Sm.

16. A method for preparing a radiolabeled DNA carrier sequence wherein a radioisotope is located internally within the DNA sequence, which comprises the steps of:
a) synthesizing a DNA sequence in at least two parts;
b) labeling the 5′ end of one of said two parts with a radioisotope;
c) hybridizing said two parts of step b) with a sequence capable of hybridizing under stringent conditions; and
d) ligating together said hybridized two parts.

17. The method of claim 16, which further include a step e) after step d) to obtain a single-stranded radiolabeled DNA carrier, which comprises
e) separating the hybridized DNA and recovering the radiolabeled DNA carrier sequence.

18. The method of claim 12, which further include a step f) after step e) to obtain a double-stranded carrier having both strand radiolabeled, which comprises:
f) hybridizing together complementary radiolabeled single-stranded DNA carrier of step e).

19. The method of claim 18, wherein said radioisotope is selected from the group consisting of ³²P, ³³P, ¹²⁵I, ¹³¹I, ³⁵S, ¹⁹⁸AU, ⁹⁰Y, ⁸⁹SR, ¹⁸⁶Re, ⁴⁵Ca and ¹⁵³Sm.

20. The method of claim 18, wherein said two parts of step a) form an antisense sequence and said sequence capable of hybridizing of step c) is a corresponding sense sequence.

21. The method of claim 18, wherein said two parts of step a) form a sense sequence and said sequence capable of hybridizing of step c) is a corresponding antisense sequence.

22. Method for the prevention of uncontrolled cell proliferation in a mammal, which comprises delivering to said mammal a therapeutic substance according to claim 1 in situ where said uncontrolled cell proliferation takes place.

23. Method according to claim 22, wherein said uncontrolled cell proliferation is a restenosis following angioplasty, and said therapeutic substance is delivered by site-specific intravascular delivery.

24. Method according to claim 23, wherein the therapeutic substance is coupled to an antibody.

25. Method according to claim 22, wherein said uncontrolled cell proliferation is cancer or a malignant tumor, and said therapeutic substance is coupled to a peptide moiety.

26. Method according to claim 25, wherein said peptide moiety is selected from the group consisting of an antibody, TGFα, TGFβ, cytokines and any growth factors.


SEQUENCE LISTING (24 sequences)

SEQ ID NO: 1 - DNA, 15 bp, Artificial Sequence
  oligonucleotide
cacgttgaggggcat

SEQ ID NO: 2 - DNA, 15 bp, Artificial Sequence
  oligonucleotide
atgcccctcaacgtg

SEQ ID NO: 3 - DNA, 15 bp, Artificial Sequence
  oligonucleotide
gcccgagaacatcat

SEQ ID NO: 4 - DNA, 15 bp, Artificial Sequence
  oligonucleotide
cctcgcagtttccat

SEQ ID NO: 5 - DNA, 19 bp, Artificial Sequence
  oligonucleotide
atgcccctcaacgtgaaaa

SEQ ID NO: 6 - DNA, 8 bp, Artificial Sequence
  oligonucleotide
cacgttga

SEQ ID NO: 7 - DNA, 7 bp, Artificial Sequence
  oligonucleotide
ggggcat

SEQ ID NO: 8 - DNA, 18 bp, Artificial Sequence
  oligonucleotide
aaaaaaaaaaaaaaattt

SEQ ID NO: 9 - DNA, 18 bp, Artificial Sequence
  oligonucleotide
tttttttttttttttaaa

SEQ ID NO: 10 - DNA, 18 bp, Artificial Sequence
  oligonucleotide
cccccccccccccccggg

SEQ ID NO: 11 - DNA, 32 bp, Artificial Sequence
  oligonucleotide
ccgcgacgatgcccctcaacgttaccatcacc

SEQ ID NO: 12 - DNA, 11 bp, Artificial Sequence
  oligonucleotide
aaaaaaaattt

SEQ ID NO: 13 - DNA, 7 bp, Artificial Sequence
  oligonucleotide
aaaaaaa

SEQ ID NO: 14 - DNA, 21 bp, Artificial Sequence
  oligonucleotide
aaatttttttttttttttccc

SEQ ID NO: 15 - DNA, 11 bp, Artificial Sequence
  oligonucleotide
ttttttttaaa

SEQ ID NO: 16 - DNA, 7 bp, Artificial Sequence
  oligonucleotide
ttttttt

SEQ ID NO: 17 - DNA, 21 bp, Artificial Sequence
  oligonucleotide
tttaaaaaaaaaaaaaaaccc

SEQ ID NO: 18 - DNA, 11 bp, Artificial Sequence
  oligonucleotide
ccccccccggg

SEQ ID NO: 19 - DNA, 7 bp, Artificial Sequence
  oligonucleotide
ccccccc

SEQ ID NO: 20 - DNA, 21 bp, Artificial Sequence
  oligonucleotide
cccgggggggggggggggaaa

SEQ ID NO: 21 - DNA, 14 bp, Artificial Sequence
  oligonucleotide
acgttaccatcacc

SEQ ID NO: 22 - DNA, 18 bp, Artificial Sequence
  oligonucleotide
ccgcgacgatgcccctca

SEQ ID NO: 23 - DNA, 35 bp, Artificial Sequence
  oligonucleotide
ggtgatggtaacgttgaggggcatcgtcgcggaaa

SEQ ID NO: 24 - DNA, 32 bp, Artificial Sequence; identical to SEQ ID NO: 11
  Oligonucleotide